(19) United States
(12) Patent Application Publication
(10) Pub. No.: US 2010/0168628 A1
(43) Pub. Date: Jul. 1, 2010

(54) HEAD FLEXION SUPPORT DEVICE

(76) Inventor: Michael Hopfenspirger, Greenwood, MN (US)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 12/650,402

(22) Filed: Dec. 30, 2009

Related U.S. Application Data
(60) Provisional application No. 61/141,405, filed on Dec. 30, 2008.

(51) Int. Cl. A61F 5/00

(52) U.S. Cl. 602/18

(57) ABSTRACT

A flexible head support worn between the chin and the chest to support the head of the wearer and reduce the possibility of neck strain or injury. The head support allows the wearer to freely move their head in all but the forward flexion direction. Adjustable straps are attached to the support in order to secure the support to the wearer. When properly worn, the head support provides a secure rest for the head of the wearer, reducing strain on the neck caused by the weight of the head when performing tasks requiring prolonged neck flexion. A central hole may be provided in the support to reduce the possibility of airway injury during use.

### RELATED APPLICATIONS

The present application claims priority to U.S. Provisional Patent Application No. 61/141,405, entitled “Device Offering Neck Flexion Support,” filed Dec. 30, 2008, the disclosure of which is hereby incorporated by reference in its entirety.

### FIELD OF THE INVENTION

The present invention relates to an apparatus for supporting the head of an individual. More specifically, the present invention relates to a device worn between the chin and the chest in order to support the head of the wearer and reduce the possibility of neck strain or injury.

### BACKGROUND OF THE INVENTION

Repetitive stress injuries (RSIs) are a widely recognized phenomenon afflicting workers and hobbyists alike. A comprehensive report and analysis of these injuries was completed by the U.S. Department of Labor, Occupational Safety and Health Administration (OSHA), Ergonomics Program (Federal Register Number: 64:65768-66078) in the form of an ergonomics proposal in 1999. The report is based on data demonstrating the risks of injury and the costs to employers for injured workers. RSIs involving the neck were frequently cited in this report.

Primary care physicians and physical therapists commonly treat patients with neck pain associated with repetitive stress. Numerous research articles have been published detailing the risks to office workers, nurses, X-ray technologists, dental workers, surgeons and computer users. Most individuals who have had to perform a repetitive task or maintain a fixed position with their body can likely relate to the associated muscle and joint pain such behavior is likely to cause.

Previously, in order to minimize the risk of RSIs workers were encouraged to employ standard ergonomic principles. These included changing one's posture, modifying the way one handles tools, and manipulating the environment in which one works in order to bring the body into a more natural, neutral posture. In doing so, an individual could hope to decrease the risk of RSIs and stress/strain to muscles and joints. While these methods can be helpful in eliminating some risk of RSIs, they are not a cure for all potential injuries.

The neck is composed of multiple joints (the cervical spine) and is as vulnerable as any other part of the body to RSIs and strain. Commonly, neck strain and RSIs are associated with prolonged neck flexion. Neck flexion is the action and position of bending the head forward (moving the chin toward the chest). This can result in neck pain, headaches, and less commonly but certainly more serious, cervical spine injury and associated nerve symptoms.

Aside from the standard principles of ergonomics, there is not a practical, wearable neck support system available. Various cervical spine collars can be used for patients with acute neck injuries. These include the larger, hard plastic devices such as the Miami-type rigid cervical brace, the SOMI-type brace and the Philadelphia brand cervical collar which are generally used to completely immobilize the neck after a cervical injury, fracture, surgery, or other trauma to the upper spine. Smaller examples include the standard “soft collar”, made of soft foam, used to offer limited, circumferential support around the entire neck.

While the above devices provide some support to the head, most are not practical for everyday use, and many fully immobilize the neck, thereby restricting neck rotation, lateral neck flexion and neck extension. Therefore, a need exists for a comfortable, supportive, and easily worn, and device for individuals who encounter extended periods of neck flexion.

### SUMMARY OF THE INVENTION

The present invention focuses on supporting the neck in the anterior flexed position and encouraging proper neck ergonomics. It does not offer support for, or resistance to neck rotation, lateral neck flexion (ear to shoulder) or neck extension. Indeed, these movements are left unencumbered to give the wearer greater mobility for neck movements that are less associated with RSIs (i.e., prolonged anterior neck flexion). The device is worn on the anterior side of the neck, between the chest and the chin. The device may be worn during the entire portion of the time when strain may be encountered, or only periodically as desired.

The device consists of a body or shell, roughly in the shape of a curved oval or saddle, with a center aperture to accommodate the anatomy of the airway and reduce the risk of possible injury to the airway. The device may be worn spaced slightly away from the skin, or positioned directly against the skin. Padding may be included on the inward-oriented side of the device to provide a cushion against the skin. The padding may be tailored with thicker portions at areas of maximal skin contact and weight support. The shell is curved in its natural position (concave to the outward direction) to follow the anatomical topography of the anterior neck. The device is securely held in place with a soft, woven elastic strap that is worn around the back and sides of the neck and attaches to the device on either end. The strap may be adjustable and detachable.

The strap attaches on each end to an additional piece of flexible material such as plastic with 2-point fixation to the saddle, offering stability to the entire device. The device is easily fastenable and removable.

In one embodiment of the invention, the device offers minimal resistance to limited anterior neck flexion and increases the amount of resistance as the degree of neck flexion increases, such that the more downward force the wearer exerts on the device the greater the amount of support the device will provide. Therefore, the device accomplishes two goals. Firstly, it reminds and encourages the wearer to maintain a normal, healthy upright neck posture and thus limiting the amount of possible stress or strain the neck is exposed to. The more the wearer flexes the neck, the greater the degree of resistance. Secondly, if the wearer chooses, the lower jaw and chin can essentially rest on the device, thus resting the weight of the head and removing stress and strain on the neck.

In another embodiment, the stiffness, or amount of resistance offered to head flexion, of the device may be modified and/or adjusted. One or more characteristics of the construction of the shell may be modified, such as the thickness of the shell or the material used, to alter the stiffness of the device. In another embodiment, one or more removable braces may be added to the shell to increase the stiffness of the device, increasing resistance of the shell to bending under the load of a head of a wearer.

The device may be constructed in various sizes to accommodate the greatest number of people. Alternatively, the height or width of the device may be adjusted to accommodate various neck lengths. Further, the positioning of the device when worn may be adjusted as desired by the wearer, so as to provide greater or lesser freedom of neck flexion. For example, the device may be worn slightly lower on the chest if a wearer desires an increased range of unsupported neck flexion, or the device may be work higher on the chest if the wearer desires a greater amount of head support.

The device may be customizable, either in manufacture or by an end user, so as to alter the look of the device and/or strap with various colors, logos, and/or patterns. Suitable applications for the device are many, including but not limited to: surgeons, nurses and other personnel in an operating room, dentists, ice fishermen, assembly line workers, factory workers, and computer operators.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of one embodiment of the device.

FIG. 2 is a view of the anterior side of an embodiment of the device.

FIG. 3 is a side view of an embodiment of the device.

FIG. 4 is a view of the posterior side of an embodiment of the device.

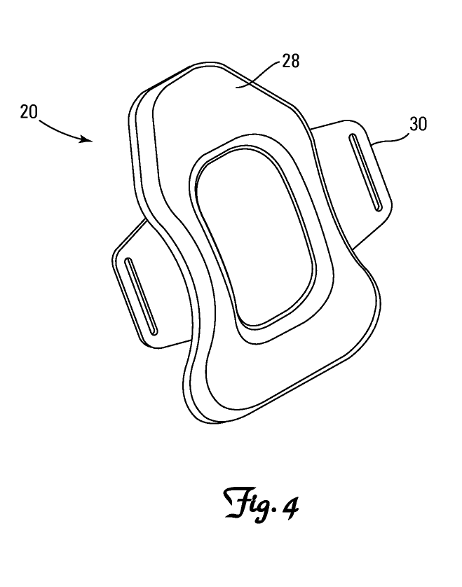

FIG. 5 is a perspective view of another embodiment of the device.

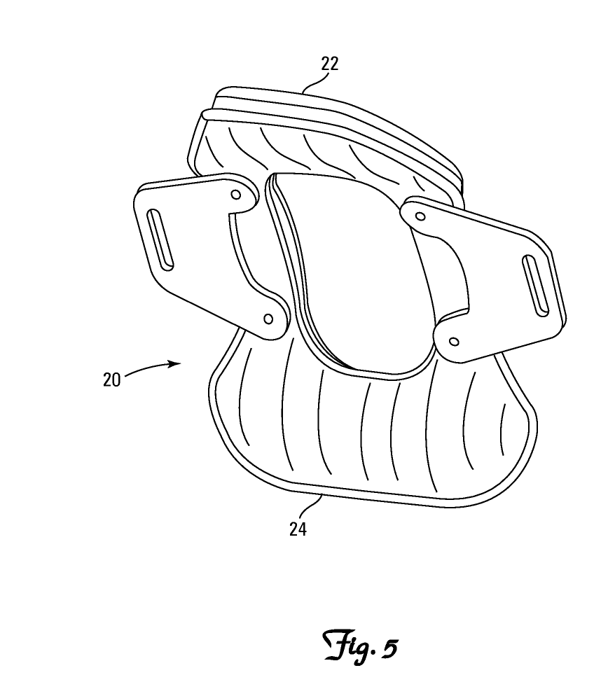

FIG. 6 is a side view of another embodiment of the device.

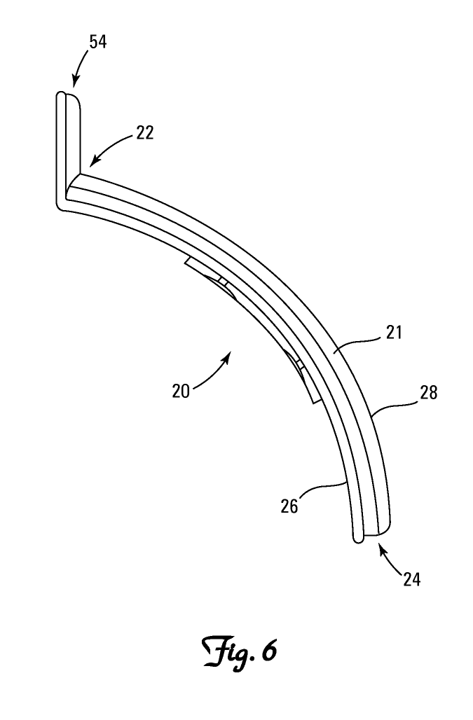

FIG. 7A is a perspective view of another embodiment of the device.

FIG. 7B is a side view of another embodiment of the device.

FIG. 7C is a side view of another embodiment of the device.

FIG. 8A is a side view of a person wearing an embodiment of the device.

FIG. 8B is a side view of a person wearing an embodiment of the device, with the chin of the person resting on the device.

FIG. 8C is a further side view of a person wearing an embodiment of the device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

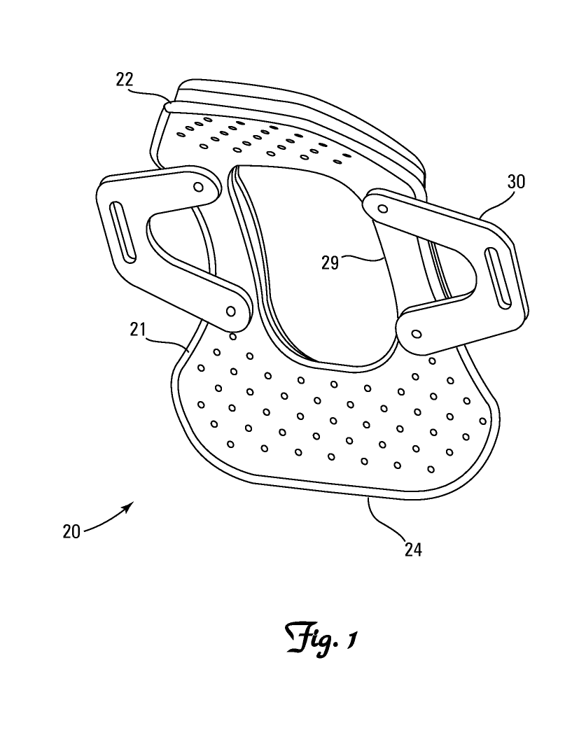

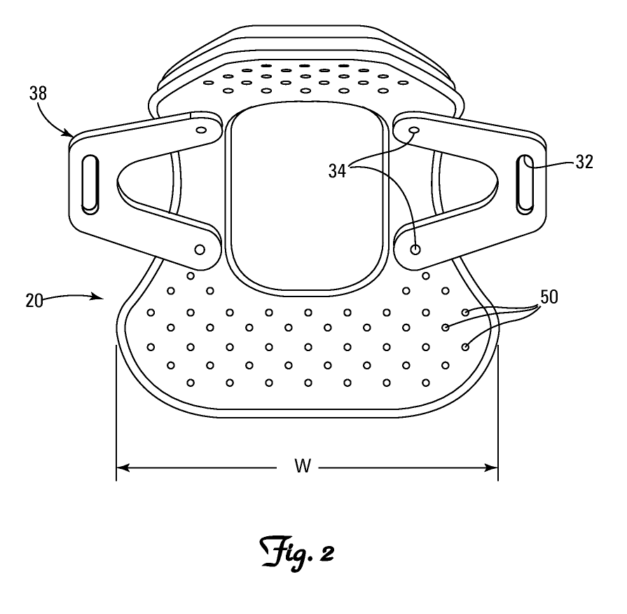

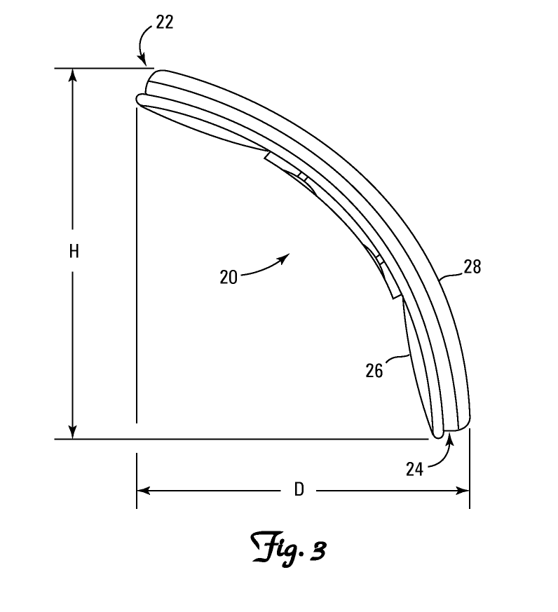

Referring generally to FIGS. 1-3, one embodiment of head support device 20 is depicted. Device 20 generally includes a body shell 21 having a first end 22, a second end 24, an anterior side 26 and a posterior side 28. Body shell 21 is generally saddle-shaped, being curved in the superior-inferior direction. Body 21 may be constructed of various polymers that are generally rigid, but still somewhat flexible to provide a desired amount of flex under load. In one embodiment, the device may be completely rigid. Body 21 may be constructed of an elastic material, such that it will return to its original shape after supporting a head of a person. Plastics are desirable for the construction of shell 21, due to low cost and ease of manufacturing, however, other materials are within the scope of the invention such as fiberglass, Kevlar, carbon fiber, or other similar materials.

First end 22 is configured to be in contact with the chin of a wearer of device 20, and second end 24 is configured to be in contact with the chest of the wearer. When positioned on a person, first end 22 is oriented superior, second end 24 oriented inferior, with anterior side 26 facing outward away from the wearer and posterior side 28 facing inward toward the neck of the wearer. As seen generally in the Figures, anterior side 26 is concave while posterior side 28 is convex. Second end 24 may be wider than first end 22, to provide a wide area for contact with the wearer's chest or upper torso. A comparatively narrow first end 22 provides ease of head extension, rotation and lateral flexion, while still providing sufficient support for head flexion. The first end 22 is generally referred to herein as being configured to contact the chin of the wearer, but it should be understood that first end 22 may contact the lower jaw or upper neck with the same result. In one embodiment, body 21 is provided with a chin-stop feature 54 on first end 22. Chin-stop 54 provides an additional surface for a wearer to more comfortably rest their chin on while using device 20.

Device 20 may include an aperture 29, located generally in the middle of body 21. Aperture 29 allows provides clearance for the anatomy of the airway and reduces the possibility of discomfort or injury to the airway while wearing device 20. Aperture 29 allows device 20 to fit snugly on the neck of a wearer, if desired, without discomfort. Head support device 20 may include padding 52, especially on or proximate first end 22, second end 24, and/or posterior side 28. The padding 52 may be tailored with thicker portions at areas of maximal skin contact and weight support. Ventilation may be included in device 20, such as in the form of air holes 50.

In one embodiment, head support device 20 can be used by simply placing second end 24 into contact with the chest of a person, and lowering the chin onto first end 22, such that the weight of the head of the person keeps device 20 in place. In other embodiments, one or more retention means 38 can be used with body 21 to securely hold the device in place on the person. Such retention means 38 may are preferably adjustable to suit a variety of users, and may include woven or artificial straps or bands, with or without elastic, or other such arrangements known in the art. Various fastening means may be used to secure the retention means to body 21, such as mechanical fasteners, hook-and-loop fasteners, adhesives, buckles, snaps and so forth.

In one embodiment, shell 21 is provided with a plurality of strap attachments 30, which may be an integral part of shell 21, or coupled to shell 21 at points 34 with a variety of suitable fasteners known in the art. Each strap attachment 30 includes a slot 32 for receiving a suitable strap 36.

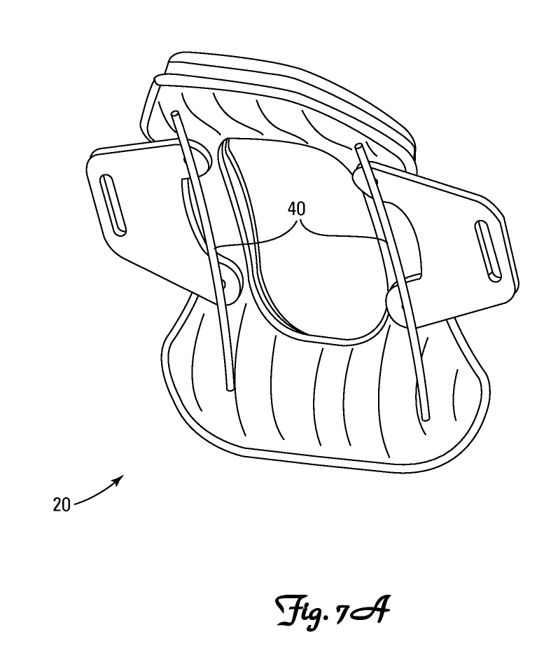

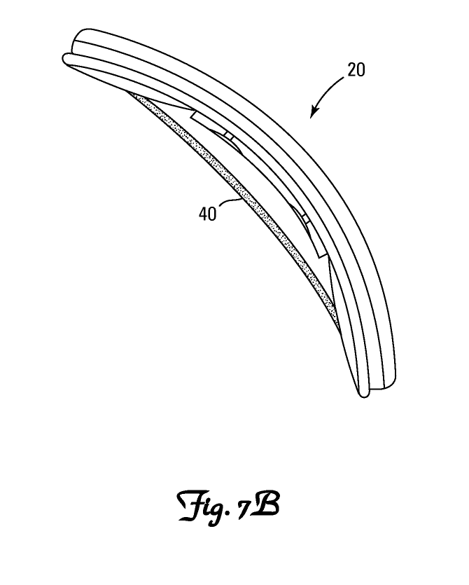

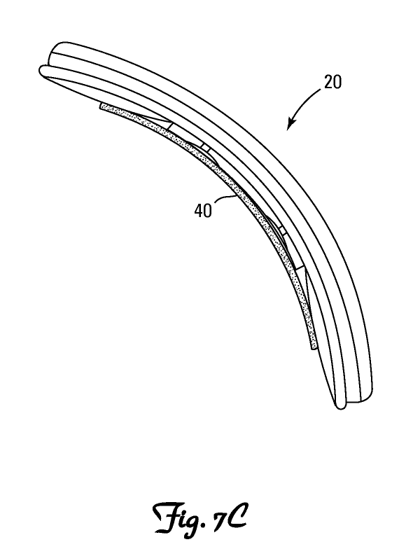

In another embodiment, the stiffness or amount of resistance offered, of device 20 may be modified, customized and/or adjusted. One or more characteristics of the construction of the shell may be modified, such as the thickness of the shell or the material used, to alter the stiffness of the device. In another embodiment, one or more braces 40 may be added to shell 21 to increase the stiffness of device 20, increasing resistance of the shell to bending under the load of a head of a wearer, such as depicted in FIGS. 7A-7C. Brace 40 may be constructed of a material similar to that used for body 21, or of a stiffer material. Braces 40 may be in the form of bars, or leaves, or other suitable shapes. Brace 40 may be coupled to shell 21, or intergrally formed with the shell. These braces 40 may be attached in a variety of positions to the shell 21 and configured in a variety of shapes and thicknesses to achieve the desired amount of flex and resistance desired by the wearer. In one embodiment, braces 40 may be configured with features for attaching a retention means 38 thereto.

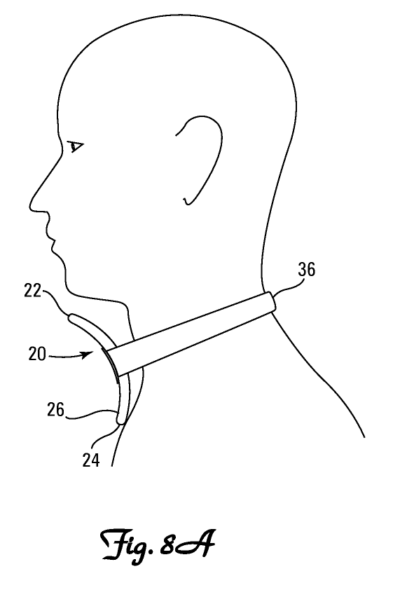

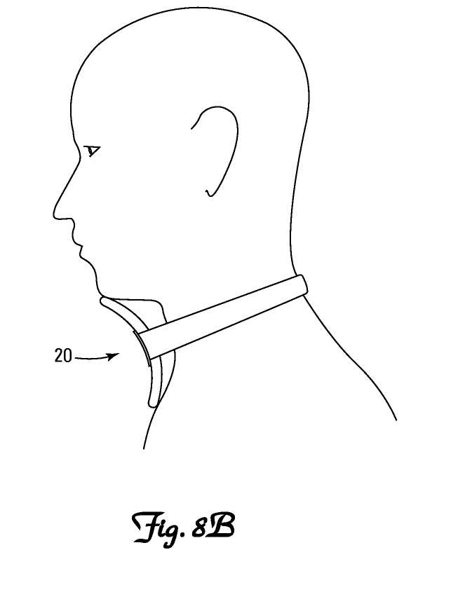

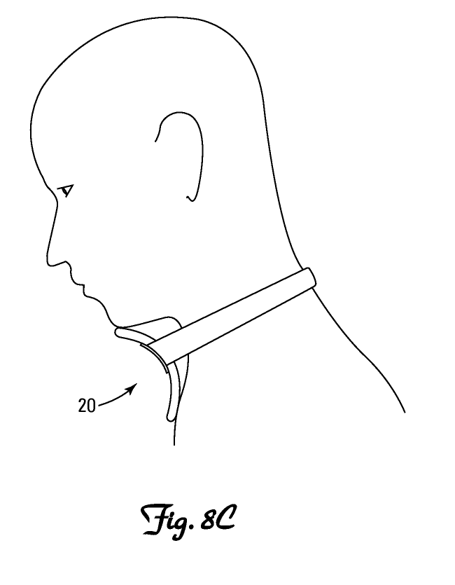

Referring now to FIG. 8A, device 20 is depicted on a wearer, with second end 24 resting on the chest and strap 36 around the neck of the wearer. Device 20 is configured and adjusted to allow a slight amount of head flexion without support from device 20, as can be seen by the space between first end 22 and the chin of the wearer. Additionally, head extension, rotation and lateral flexion movements are unencumbered with the device on. In FIG. 8B, the wearer has lowered his head slightly onto device 20, such that his chin is resting on first end 22. FIG. 8C depicts an embodiment wherein device 20 is configured to flex under the weight of a wearer's head. Increased head flexion from the wearer has resulted in the concavity of the device being greater than in FIG. 8B.

The device 10 may be made in various sizes to accommodate the greatest number of people. Alternatively, the height or width of the device may be adjusted to accommodate various neck lengths. By way of example, in one embodiment device 20 has a height H of approximately 18 cm, a width W of approximately 11 cm, a depth D of approximately 10 cm, and a thickness of approximately 1.5 cm.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the inventions may comprise fewer features than illustrated in any individual embodiment described above. The embodiments described herein are not meant to be an exhaustive presentation of the ways in which the various features of the inventions may be combined. Accordingly, the embodiments are not mutually exclusive combinations of features; rather, the inventions may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the embodiments of the present inventions, it is expressly intended that the provisions of Section 112, sixth paragraph of 35 U.S.C. are not to be invoked unless the specific terms “means for” or “step for” are recited in a claim.

1. A head flexion support apparatus, comprising:
a body including a first end, a second end, and a generally concave anterior profile extending between the first end and the second end, the first end configured to interface with a chin of a person and the second end configured to interface with a chest area of the person.

2. The apparatus of claim 1, wherein the body is generally rigid in a superior-inferior direction.

3. The apparatus of claim 1, wherein the body is configured to provide increased resistance in response to an increased amount of neck flexion from a person wearing the apparatus.

4. The apparatus of claim 1, further comprising a brace removably coupled to the body, the brace configured to increase the stiffness of the body in a superior-inferior direction.

5. The apparatus of claim 4, wherein the brace is removably coupled to an anterior side of the body.

6. The apparatus of claim 1, wherein the body further comprises a generally centrally-located aperture configured to provide clearance for a laryngeal prominence of a person wearing the apparatus.

7. The apparatus of claim 1, wherein the body further comprises a stop proximate the first end configured to prevent a chin of the patient from sliding off of the apparatus.

8. The apparatus of claim 1, wherein a longitudinal axis is defined between the first end and the second end and a lateral axis is transverse to the longitudinal axis, and wherein the first end of the body is narrower laterally than the second end of the body.

9. The apparatus of claim 1, further comprising a retention strap coupled to the body.

10. The apparatus of claim 1, further comprising a means for securing the body to a person.

11. A resilient head flexion support apparatus, comprising:
an anteriorly concaved body having a first end configured to interface with a head of a person and a second end configured to interface with a chest of a person, the body flexible from a resting position to a supporting position in response to the weight of a head of a person resting on the first end, wherein the concavity of the body is greater in the supporting position than in the resting position.

12. The apparatus of claim 11, further comprising a retention strap coupled to the body.

13. The apparatus of claim 11, further comprising a means for securing the body to a person.

14. The apparatus of claim 11, wherein the body is configured to provide increased resistance in response to an increased amount of concavity.

15. The apparatus of claim 11, wherein the body further comprises a stop proximate the first end configured to prevent a chin of the patient from sliding off of the apparatus.

16. The apparatus of claim 11, further comprising a brace removably coupled to the body, the brace configured to increase the stiffness of the body in a superior-inferior direction.

17. The apparatus of claim 16, wherein the brace is removably coupled to an anterior side of the body.

18. The apparatus of claim 11, wherein the second end is wider than the first end.

19. A method, comprising:
providing a neck support device having:
a body including a first end, a second end, and a generally concave anterior profile extending between the first end and the second end; and
a retention strap coupled to the body;
providing instructions for using the neck support device to support a head of a person in a flexed position while allowing neck rotation, extension and lateral flexion, the instructions including:
securing the device to the patient with the retention strap such that the second end of the device is in contact with a chest of the person and the first end of the device is proximate a chin of the person; and
moving the head of the person such that the chin is contact with the first end of the device.

20. The method of claim 19, further comprising providing a concave neck support device movable from a first position to a second position in response to moving the head of the person such that the chin is contact with the first end of the device, wherein the concavity of the body is greater in the second position than in the first position.
